(19) United States
(12) Patent Application Publication
(10) Pub. No.: US 2020/0170994 A1
(43) Pub. Date: Jun. 4, 2020

(54) SUBLINGUAL CANNABINOID COMPOSITIONS

(71) Applicants: Yoram SELA, Ra'ananna (IL); ALVIT LCS PHARMA LTD., Ness Ziona (IL)

(72) Inventors: Yoram SELA, Ra'ananna (IL); Itschak LAMENSDORF, Modiin (IL); Nachshol COHEN, Netanya (IL)

(21) Appl. No.: 16/613,194

(22) PCT Filed: May 11, 2018
(86) PCT No.: PCT/IB2018/053307

§ 371 Date: Nov. 13, 2019

Related U.S. Application Data
(60) Provisional application No. 62/505,873, filed on May 13, 2017.

(51) Int. Cl. A61K 31/352; A61K 47/38; A61K 31/125; A61K 9/00; A61K 47/36; A61K 31/485; A61K 31/4525; A61K 31/05; A61K 47/32; A61K 31/355; A61K 31/137

(52) U.S. Cl. CPC A61K 31/352; A61K 47/38; A61K 31/125; A61K 9/006; A61K 47/36; A61K 2236/37; A61K 31/4525; A61K 31/05; A61K 47/32; A61K 31/355; A61K 31/137; A61K 31/485

(57) ABSTRACT

The present invention provides novel sublingual muco-adhesive cannabinoid compositions in the form of sublingual tablets or films, exhibiting improved bioavailability and stability, reduced side-effects such as irritation, and optionally lower dosage for the same therapeutic effect, in comparison with an alcohol-based oral spray having 5.2 mg of cannabinoid active ingredients per spray.

### FIELD OF THE INVENTION

The present invention relates to novel compositions comprising cannabis botanical extracts, isolated or pure molecules, and synthetic derivatives in a sublingual dosage form exhibiting improved bioavailability, faster onset and reduced side-effects.

### BACKGROUND OF THE INVENTION

Sublingual delivery refers to the pharmacological route of administration by which drugs diffuse into the blood through tissues under the tongue. Pharmaceuticals which have thus far been developed for sublingual administration include: cardiovascular drugs, steroids, barbiturates, enzymes, vitamins and minerals.

When an active comes in contact with the mucous membrane beneath the tongue, it diffuses through it. Advantageously, because the connective tissue beneath the epithelium contains a profusion of capillaries, the substance then diffuses into these capillaries and enters the venous circulation. In contrast, substances absorbed in the intestines are subject to “first pass metabolism” in the liver before entering the general circulation, this being a major cause for low bioavailability of known GI delivered dosage forms.

Sublingual administration has other advantages over GI administration. Being more direct, sublingual delivery may be faster acting, ensuring that the substance risks degradation only by salivary enzymes before entering the bloodstream. In contrast, swallowed drugs (upper GI delivery) must survive passage through the hostile environment of the gastrointestinal tract, risking degradation by stomach acid, bile, or one of the many enzymes therein, such as monoamine oxidase (MAO). Furthermore, as mentioned briefly above, following absorption through the gastrointestinal tract, drugs must pass through the liver, where they may be extensively altered; this is known as the first pass effect of drug metabolism. Therefore, oral or upper GI delivery is often very inefficient and hence unsuitable for some of the most important drugs widely used by patients. Cannabis is one example.

As a result of the more effective absorption that sublingual delivery can effect, it is in some cases possible to reduce the absolute dosage of the drug when sublingually administered.

Several options of sublingual administration include: regular or fast-disintegrating sublingual tablets, lipid matrix sublingual tablets, thin films and sublingual sprays. Unfortunately many of the known sublingual delivery systems suffer from the disadvantages that delivery performance and bioavailability are affected by the physical properties of the active, like solubility, crystal morphology, particle size, hygroscopicity, compressibility and mainly polarity.

Cannabis is a genus of flowering plants, sometimes divided into additional subspecies like, Cannabis indica and Cannabis ruderalis. These three taxa have long been used for fibre (hemp), seed and seed oils, medicinal purposes, and as a recreational drug. Industrial hemp products are made from cannabis plants selected to produce an abundance of fiber. In addition specific cannabinoids were isolated from the full extract, mainly THC and CBD,

Cannabis strains have been bred to produce desired levels of THC and/or CBD. In some cases, having minimal levels of THC, the principal psychoactive constituent obtained through the dried flowers of cannabis plants, and in other cases to produce high levels of THC and other psychoactive cannabinoids. Various extracts including hashish and hash oil are also produced from the plant.

Nabiximols® (also referred to hereinbelow by its USAN trade name Sativex®) is a patented cannabinoid oromucosal mouth spray developed by the UK company GW Pharmaceuticals for multiple sclerosis (MS) patients, who can use it to alleviate neuropathic pain, spasticity, overactive bladder, and other symptoms. It is also approved in Canada, France and some other European countries for some of the above-mentioned indications. Nabiximols® is also being developed as a potential treatment to alleviate pain associated with cancer, and it has also been researched in various models of peripheral and central neuropathic pain.

Nabiximols® is distinct in that it contains a mixture of compounds derived from cannabis plants, rather than a single molecular synthetic product. Although it is a pharmaceutical product standardized in composition, formulation and dose, Sativex® is still in essence a tincture of the cannabis plant and its principal active cannabinoid components are the cannabinoids: tetrahydrocannabinol (THC) and cannabidiol (CBD). The product is formulated as an oromucosal spray which is administered by spraying into the mouth. Each spray delivers a near 1:1 ratio of CBD to THC, with a fixed dose of 2.7 mg THC and 2.5 mg CBD.

Chemistry and Pharmacokinetics

Cannabinoid pharmacokinetics encompasses absorption via diverse routes of administration and from different drug formulations, analyte distribution throughout the body, metabolism by the liver and extra-hepatic tissues, and elimination in the feces, urine, sweat, oral fluid, and hair. Pharmacokinetic processes are dynamic, may change over time, and may be affected by the frequency and extent of drug exposure. Cannabinoid pharmacokinetics research is challenging due to low analyte concentrations, rapid and extensive metabolism, and physico-chemical characteristics hindering the separation of drugs of interest from biological matrices and from each other. More than 421 different chemical compounds, including over 60 cannabinoids, have been identified or isolated from Cannabis sativa.

Understanding cannabinoid plant chemistry has proven far more complex than simply looking at pure THC. Different effects may be experienced due to the presence or absence of additional cannabinoids and other chemicals. Eighteen different classes of chemicals, including nitrogenous compounds, amino acids, hydrocarbons, carbohydrates, terpenes, and simple and fatty acids, contribute to the known pharmacological and toxicological properties of cannabis. THC is usually present in cannabis plant material as a mixture of monocarboxylic acids, which readily and efficiently decarboxylate upon heating. THC decomposes when exposed to air, heat, or light; exposure to acid can oxidize the compound to cannabinol (CBN), a much less-potent cannabinoid. In addition, cannabis plants dried in the sun release variable amounts of THC through decarboxylation. The pyrolysis caused by smoking whole or agriculturally sourced cannabis may produce more than 2,000 compounds. Due to the chemical complexity of cannabis plant material compared to synthetic THC, extracts of cannabis that capture the full range of cannabinoids are being explored as therapeutic medications.

While cannabis has been used as medicine for thousands of years, cultivation methods have been developed in recent decades to reproducibly yield plants with defined THC or CBD profiles—i.e. concentrations and ratios.

Two standardized extract preparations are known: Tetranabinex M® which is high in THC, and Nabidiolex M®, which is high in CBD are known. Sativex M® contains nearly equal (i.e. 1:1) proportions of Tetranabinex M® and Nabidiolex M®, and hence, almost equal amounts of THC and CBD. THC and CBD comprise approximately 70% of the active ingredient of the product, with 5% comprising other cannabinoids, and the remainder comprising terpenoids, flavonoids, sterols, alkanes, and other chemicals.

As can be easily observed from FIG. 1, four sprays of the approved oral spray dosage form Sativex® shows very limited bioavailability when compared to vaporised THC. In addition since this spray is an alcoholic spray, patients with sensitive oral mucosa suffer from undesirable irritation and other topical side effects, a severe compliance issue, on top of the product's low bioavailability.

Thus there is an unmet need for an effective, reliable and reproducible sublingual delivery system, especially when dealing with poorly soluble drugs like cannabinoids.

### SUMMARY OF THE INVENTION

The exemplary embodiments of the inventive formulations and methods of treatment made possible by them, provide novel sublingual compositions such as sublingual tablets and films comprising cannabinoid active ingredients, a generic term used herein to widely refer to whole or partial cannabis botanical extracts, purified/isolated cannabinoids and synthetic derivatives of cannabinoids, whole or partial, which contains the entire range of extracted cannabinoids, including specific cannabinoids like THC, CBD and others, formulated into unique sublingual compositions comprising the cannabis botanical extract, Vitamin E TPGS (apparently functioning as an antioxidant in addition to its usual role as emulsifier and enhancer), menthol (or similar permeation enhancer) and mucoadhesive polymers including carbomer, CMC, polyethylene oxides, HPMC, HMC and polyvinylpyrollidone.

The novel compositions provide effective and stable sublingual dosage forms having mucoadhesive properties, enabling superior bioavailability of the active ingredients, thereby reducing side-effects and permitting lower doses than has hitherto been thought to be theraspeutically effective without compromising therapeutic efficacy.

The current invention provides a less or non-irritating sublingual tablet or film with mucoadhesive capabilities and improved bioavailability by including polymers like Carbomer and PVP, which result in prolonging sublingual contact and adherence, as well as permeation enhancers such as Vitamin E TPGS and menthol. In addition, the novel compositions of this invention exhibit improved stability of the cannabinoid active ingredients, due to their being in a dry form as well as, it is believed, possibly due to the presence of Vitamin E TPGS, which has antioxidant properties.

The novel sublingual compositions enable the reduction of the cannabinoid active ingredient dose, and mainly the psychoactive THC active ingredient, by 50% or more, without compromising its therapeutic efficacy as compared to Sativex®.

According to one embodiment, there is provided a method of treatment of neuropathic pain or inflammation by administration to a patient in need thereof of a therapeutically effective amount of a composition of this invention.

Said neuropathic pain or inflammation may result from chemotherapy.

In one embodiment, there is provided a method of treatment of epilepsy by administration to a patient in need thereof of a therapeutically effective amount of a composition of this invention.

According to another embodiment, there is provided a method of treatment of Parkinson disease, seizures, epilepsy, PTSD and the like by administration to a patient in need thereof of a therapeutically effective amount of a composition of this invention.

In another embodiment, there is provided a method of treatment of MS related spasm by administration to a patient in need thereof of a therapeutically effective amount of a composition of this invention.

According to one embodiment, there is provided a method of treatment of cancer-related pain or inflammation by administration to a patient in need thereof of a therapeutically effective amount of a composition of this invention.

In another embodiment, there is provided a method of treatment of a medical condition by administration to a patient in need thereof of a therapeutically effective amount of the composition of this invention.

According to one embodiment, there are provided novel sublingual adhesive compositions in the form of sublingual tablets or films, exhibiting improved bioavailability and stability, reduced side-effects such as irritation, and optionally lower dosage for the same therapeutic effect, in comparison with the commercially available product Sativex®.

### BRIEF DESCRIPTION OF THE FIGURES

FIG. 1 is a published chart showing comparative bioavailability of active ingredients delivered by the known oral spray dosage form Sativex® versus vaporized THC.

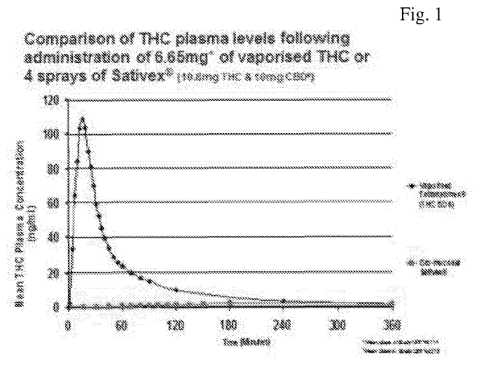

FIG. 2 is a chart showing comparative plasma levels in ng/ml of combined THC and CBD active ingredients delivered by 4 sprays of the known oral spray dosage form Sativex® versus 2 of the inventive tablet dosage forms prepared according to an exemplary embodiment of the present inventive techniques.

FIG. 3 is a table taken from the literature showing a listing of compounds which may be considered as representative cannabinoid active ingredients.

### DETAILED DESCRIPTION OF EXEMPLARY EMBODIMENTS

The wording herein below is implied in the common meaning of the definitions and statements as known to the versed in the art of pharmaceuticals and polymer science. However, there are several terms that should be understood within the context of inventive techniques, formulations, compositions and treatments as follows:

As used in the specification and claims, the forms “a”, “an” and “the” include singular as well as plural references unless the context clearly dictates otherwise.

Further, as used herein, the term “comprising” is intended to mean that the system includes the recited elements, but not excluding others which may be optional in the design of the system, such as fillers and the like. The term “consisting essentially of” is used to define a system that includes the recited elements but exclude other elements that may have an essential significance effect on the performance of the system. “consisting of” shall thus mean excluding more than traces of other elements. Embodiments defined by each of these transition terms are within the scope of this invention.

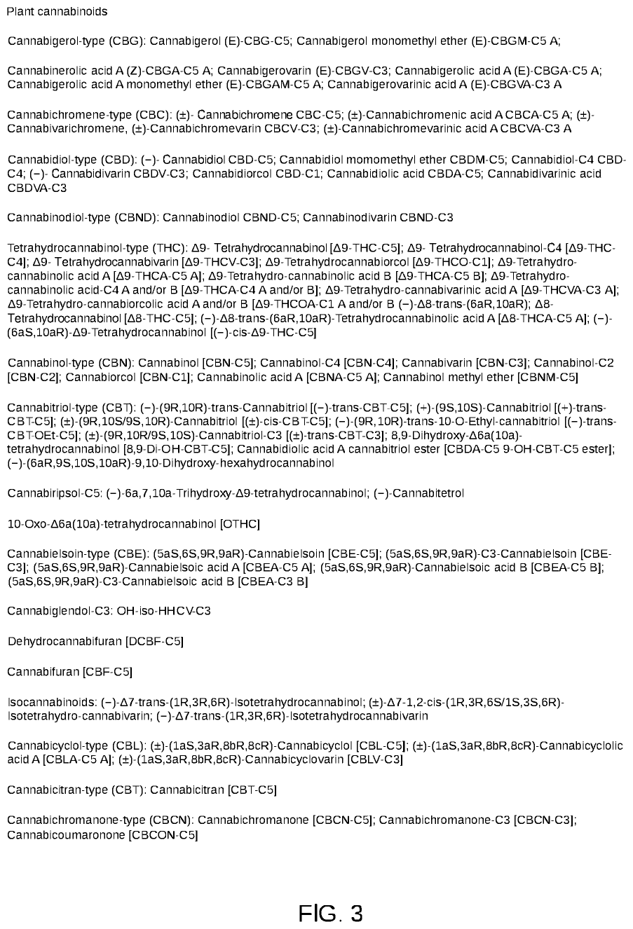

The terms “active”, “active pharmaceutical ingredient” and API relate to the pharmaceutical active materials and are used interchangeably. FIG. 3 includes a non-exhaustive list of active ingredients which will be referred to collectively herein as included within the wider and more generic term “cannabinoid active ingredients”. The term cannabinoid active ingredients should also be understood as including non-plant derived cannabinoids, cannabinoid-like molecules derived from plants and sources other than Cannabis species, and synthetic derivatives of cannabinoids.

The terms “botanical extracts” and “extracts” relate to a mixture of substances obtained from the plants by an extraction process followed by concentration, and are used interchangeably.

Following are short descriptions of exemplary excipients which may be used in the novel compositions and methods of treatment made possible by the disclosed inventive techniques.

Vitamin E TPGS NF, sourced from Eastman Co., d-α-tocopheryl polyethylene glycol 1000 succinate, is a surfactant that is used as an emulsifier, drug solubilizer, absorption enhancer, and as a vehicle for lipid-based drug-delivery formulations. Vitamin E TPGS has found wide utility in pharmaceutical formulations including the following: improvement of drug bioavailability, enhancing solubilization of poorly water-soluble drugs due to its surfactant properties, stabilization of amorphous drug forms and enhancing drug permeability by P-glycoprotein efflux inhibition.

Menthol is an organic compound made synthetically or obtained from cornmint, peppermint or other mint oils. A waxy, crystalline substance, clear or white in color, it is solid at room temperature and melts at temperatures slightly above. The main form of menthol occurring in nature is (−)-menthol, which is assigned the (1R,2S,5R) configuration. Menthol has local anesthetic and counterirritant qualities, and it is widely used to relieve minor throat irritation. Menthol may also act as a weak kappa opioid receptor agonist].

Menthol is included in many pharmaceutical products for a variety of reasons. Its pharmaceutical and medicinal uses are extensive. More relevant for the current application, menthol is useful in transdermal and transmucosal preparations as a permeation enhancer.

Mucoadhesive Polymers

The mucoadhesive polymers used in the novel compositions of this invention exhibit mucoadhesion ability and are selected from the group comprising polyethylene oxide (PEO), carbomer, polyvinylpyrrolidone (povidone, PVP), cellulose based polymers and chitosan in an amount ranging from 2-100 mg per dosage form. HEC, NaCMC, HMC are additional examples of carboxy gels that may be useful for practicing the inventive techniques and preparing the inventive mucoadhesive sublingual dosage forms. See for example Adamo F., et al., Mucoadhesive Gels Designed for the Controlled Release of Chlorhexidine in the Oral Cavity, Pharmaceutics 3, 665-679; 2011 doi:10.3390/pharmaceutics3040665 ISSN 1999-4923, Received: 21 Jul. 2011; in revised form: 9 Sep. 2011/Accepted: 26 Sep. 2011/Published: 27 Sep. 2011. This reference describes the use of carboxymethyl-(CMC), hydroxypropylmethyl-(HPMC) and hydroxypropyl-(HPC) cellulose, alone (3% w/w) or in binary mixtures (5% w/w) using chlorhexidine as the active ingredient.

Carbomer (carboxy vinyl polymer also known as carbopol) is a very high molecular weight polymer of acrylic acid with cross linkages of allyl sucrose. Due to the high proportion of the carboxy groups present, carbomer solution is known to be acidic. It is also of low viscosity but when neutralized with triethanolamine, it is converted to highly viscous gels. The adhesive properties of carbomer are exploited to develop mucoadhesive gels and drug delivery systems for controlled and localized drug delivery.

Carbopol polymers have been used worldwide for many years to thicken, modify flow characteristics, emulsify, and suspend insoluble ingredients. Recently, interest in their mucoadhesive properties has grown dramatically.

Mucoadhesion (or muco-adhesion) is generally understood to define the ability of a biological or synthetic material to “stick” to a mucous membrane, resulting in bioadhesion of the material to the tissue for a protracted period of time. This concept has received a significant degree of attention, due to potential applications in drug delivery and enhanced drug bioavailability which results from the lengthened period of time in which the mucoadhesive dosage form is in contact with the absorbing tissue versus a standard dosage form. In order for a material to be mucoadhesive, it must interact with mucus, which is a highly hydrated, viscous anionic hydrogel layer protecting the mucosa. The mucin is composed largely of flexible glycoprotein chains, which are crosslinked. Carbomer is very efficient at this task.

Preparation of Botanical Cannabis Extracts

The two main cannabinoids present in the various cannabis strains are tetrahydrocannabinol (THC) and cannabidiol (CBD).

Certain cannabis taxa and strains contain these two cannabinoids in various percentages and ratios.

The novel compositions of this invention are prepared from several types of cannabis Botanical Extracts, comprising the two cannabinoids THC and CBD in various ratios, according to therapeutical needs.

The above extracts are obtained either by an extraction process using super critical fluid method (CO 2) extraction, or nonpolar extraction with butane from plant strains producing THC and CBD in specific and reproducible ratios.

Thus, while clones of cannabis strains CN1 are producing about equal amounts of CBD and THC, cannabis strain MM9 produces mostly THC, and HB3 produces mostly CBD. The preparation of the cannabis botanical extracts from these strains is described in Examples 1-6 below.

Preparation of the Novel Sublingual Mucoadhesive Compositions

The novel sublingual adhesive compositions of this invention in the form of tablet or film are prepared from cannabis botanical extracts of various THC/CBD ratios or pure cannabinoids, Vitamin E TPGS, menthol (crystals or oil), a mucoadhesive polymer (carbomer, PVP, and most hydrogels i.e. those with mucoadhesive characteristics, and other pharmaceutically acceptable inactive ingredients.

The novel compositions comprise cannabis botanical extracts, cannabis isolates, such as purified CBD or THC, their derivatives whether obtained by pyrolysis or entirely synthetic cannabinoids, in a therapeutically effective dose, which is likely to be something significantly less than contained in daily doses of Sativex®.

Examples 1-6 below detail the preparation of compositions comprising a total of between 5-20 mg of the two cannabinoids THC and CBD in various ratios.

For comparison, Sativex® contains a near 1:1 ratio of THC to CBD and is administered as a spray. Each spray puff delivers a fixed dose of 2.7 mg THC and 2.5 mg CBD (a total of 5.2 mg cannabinoids/puff). The treatment includes 5-13 daily puffs, that is 26-67.6 mg cannabinoids THC and CBD/day.

Thus, the compositions of the instant invention contain lower doses of THV/CBD per tablet or film than those used in the Sativex® treatment.

Due to the unique properties of the novel compositions, it is expected that dosages of the novel compositions which are lower than those of the commercially available product Sativex® will lead to comparable or better therapeutic effects.

In one novel application of the exemplary embodiments of the present inventive techniques, one or more additional active ingredient, here buprenorphine HCl and/or naloxone) may be added to the compositions. The combination tablets contain 10 mg of THC/CBD 1:1 and 8 mg buprenorphine HCl.

The preparation of the novel sublingual sublingual compositions is described in Examples 1-6 below.

According to one embodiment, there are provided compositions comprising a therapeutically effective dose of a botanical extract of cannabis, pure cannabinoid isolates or synthetic cannabinoid derivatives, Vitamin E TPGS, menthol, a mucoadhesive polymer, other pharmaceutically acceptable ingredients and optionally an additional active, wherein the composition is administered sublingually in the form of a tablet or film and is mucoadhesive.

The cannabis botanical extracts of this invention comprise between 5-95% THC (tetrahydrocannabinol) and between 5-95% CBD (cannabidiol).

Said cannabis botanical extract may be extracted from the proper cannabis plant strain by an extraction method selected from supercritical fluid extraction with CO2 and extraction with a non-polar solvent.

According to one exemplary embodiment, the sublingual composition of this invention comprises a muco-adhesive excipient.

The mucoadhesive polymer used in the compositions exhibits mucoadhesion ability and is selected from the group comprising PEO, carbomer, PVP, cellulose based polymers and chitosan in an amount ranging from 2-100 mg per dosage form.

Vitamin E TPGS used in the compositions plays the combined role of stabilizer, permeation enhancer and solubilizer. Using TPGS results in a self-emulsified dosage form. The same effect can be achieved with minimal experimentation by one of ordinary skill in the art by using other surface active materials that lack the permeation enhancement activity of the TPGS in combination with a separate though somewhat less versatile permeation enhancer.

The novel composition may further comprise an antioxidant selected from BHT, BHA and their mixtures.

The sublingual mucoadhesive compositions comprise menthol as oil or crystals in an amount of 1-50 mg per dosage form.

According to one embodiment, there is provided a method of treatment of opioid addiction and dependency by administration to a patient in need thereof of a therapeutically effective amount of a composition of this invention, comprising a therapeutically effective dose of a botanical extract of cannabis and optionally an additional active. One example of an optionally added active ingredient may be buprenorphine HCl. Buprenorphine HCl and the two actives CBD and THC work synergistically as an anti-opioid anti-addiction and dependency combination product.

### EXEMPLARY EMBODIMENTS

The following examples further illustrate the invention as it may be carried out but, of course, should not be construed as in any way limiting its scope. The scope of the inventive techniques should of course be only understood as limited to and with reference to the claims.

### Example 1

Cultivation of the Plant Material

Cultivate cannabis clones of strains CN1 (who is producing about equal amounts of CBD and THC), MM9 (who is producing mostly THC), and HB3 (who is producing mostly CBD), in a soil-less growing medium consisting of 50%-50% coco coir and perlite with a fertigation system using pure food grade mineral fertilizers. The cultivation is carried out in a greenhouse with climate control according to standardized growing protocol so as to produce a consistent chemical profile over several seasons. No chemical pesticides are to be used for pest control, in order to avoid residues in the end product. Use an integrated pest management system, consisting of mechanical separation using double entries with 50 mesh insect nets, natural oils application and spreading biological pest control selected from Phytoseiulus persimilis, Diglyphus isaea, Aphidius colemani, Nesidiocoris tenuis, Cryptolaemus montrouzieri. Determine harvesting time by organoleptic testing via X100 field microscope examining trichromes colour and structure. Use further examination with a HPTLC field semi-quantitative chemical analysis kit for initial QC. Carry out harvesting by cutting the whole plant from the main stem and hanging the plant upside down on plastic wires in a temperature and humidity controlled dark room at 20 degrees Celsius and 50%-60% relative humidity using fans to create air movement in order to prevent grey mold. Strip the plants off their stems once plants have reached 10%-12% moisture content upon loss on drying, and store floral and leaf material in aluminum light proof vacuum bags as the extraction plant raw material.

Preparation of the THC:CBD 1:1 Cannabis Extract

Using a Cannabis sativa clone containing about equal amounts of THC (tetrahydrocannabinol) and CBD (cannabidiol), grown under GAP conditions as determined by WHO as source of the botanical extract.

Dry, mill and then extract the plants by using super critical fluid method (CO2) extraction, or non polar extraction with butane then concentrate the botanical extract to about 70% w/w concentration of THC:CBD 1:1. This THC/CBD 1:1 70% extract is defined as the THC/CBD 1:1 cannabis extract in this application and is used in the sublingual preparations of this invention.

Preparation—Sublingual Mucoadhesive Cannabis Extract Tablets Containing 1:1 THC/CBD

Mucoadhesive Sublingual Tablets Preparation:

Blend the THC/CBD 1:1 cannabis extract (200 gr of the 70% w/w mixture) together with 800 gr mannitol/lactose 1:1 mixture, 30 gr PVP K-30, 20 gr Vitamin E TPGS, granulate with 600 gr ethanol USP and then dry for 45 minutes in a Glatt fluidized bed dryer, at inlet temp 50 deg C.

Mix the dry mixture with an additional amount of 200 gr mannitol, 10 gr sodium citrate & citric acid, 3 gr lemon flavor, 10 gr carbomer 934, 20 gr corn starch and 10 gr magnesium stearate. Prepare sublingual THC/CBD 1:1 cannabis extract tablets containing 10 mg of THC/CBD from the above mixture.

### Example 2

Preparation of the THC/CBD 9:1 Cannabis Extract

Use Cannabis sativa clone containing about 90% of THC and 10% CBD, grown under GAP conditions as determined by WHO as source of the botanical extract.

Dry, mill and then extract the plant by using super critical fluid (CO2) extraction, or non polar extraction with butane. then concentrate the botanical extract to about 70% w/w concentration of THC &CBD (which are in ratio of about 9:1).

This THC/CBD 9:1 70% extract is defined as the THC/CBD 9:1 cannabis extract in this application and to be used in the sublingual preparations.

Preparation of Sublingual Mucoadhesive Tablets Containing THC/CBD 9:1 Cannabis Extract

Mucoadhesive Sublingual Tablets Preparation:

Blend the THC/CBD 9:1 cannabis extract (100 gr of the 70% w/w mixture) together with 800 gr mannitol/lactose 1:1 mixture, 30 gr PVP K-30.20 gr Vitamin E TPGS, 10 gr crystalline menthol, granulate with 600 gr ethanol USP and then dry for 45 minutes in a Glatt fluidized bed dryer, at inlet temp 50 deg C.

Mix the dry mixture with an additional amount of 200 gr mannitol, 10 gr sodium citrate & citric acid, 3 gr lemon flavor, 10 gr carbomer 934, 20 gr corn starch and 10 gr magnesium stearate.

Prepare sublingual THC/CBD 9:1 cannabis extract tablets containing 5 mg of THC/CBD 9:1 from the above mixture.

### Example 3

Preparation of the THC/CBD 1:9 Cannabis Extract

Using Cannabis sativa clone containing about 10% THC and 90% of CBD, grown under GAP conditions as determined by WHO as source of the botanical extract.

Dry, mill and then extract the plant by using supercritical fluid method (CO2) extraction, or nonpolar extraction with butane. Concentrate the botanical extract to about 70% w/w concentration of THC/CBD (which are in ratio of 1:9). This THC/CBD 1:9 70% extract is defined as the THC/CBD 1:9 cannabis extract in this application and is to be used in the sublingual preparations.

Preparation of Sublingual Mucoadhesive Tablets Containing THC/CBD 1:9 Cannabis Extract

Mucoadhesive Sublingual Tablets preparation:

Blend the THC/CBD 1:9 cannabis extract (100 gr of the 70% w/w mixture) together with 800 gr mannitol/lactose 1:1 mixture, 30 gr PVP K-30, 20 gr Vitamin E TPGS, 10 gr crystalline menthol, then granulate with 600 gr ethanol USP and dry for 45 minutes in a Glatt fluidized bed dryer, at inlet temp 50 deg C.

Mix the dry mixture with an additional amount of 200 gr mannitol, 10 gr sodium citrate & citric acid, 3 gr lemon flavor, 10 gr PEO 301, 20 gr corn starch and 10 gr magnesium stearate.

Prepare sublingual THC/CBD 1:9 cannabis extract tablets containing 5 mg of THC/CBD 1:9 from the above mixture.

### Example 4

Preparation of Mucoadhesive Sublingual Combination of THC/CBD 1:1 Cannabis

Extract and Buprenorphine

Using cannabis Sativa clone containing about equal amounts of THC (tetrahydrocannabinol) and CBD (cannabidiol), grown under GAP conditions as determined by WHO as source of the botanical extract.

Dry, mill and then extract the plant by using super critical fluid method (CO2) extraction, or non-polar extraction with butane. Concentrate the botanical extract is to about 70% w/w concentration of CBD &THC (which are in ratio of about 1:1). This THC/CBD 1:1 70% extract is defined as the THC/CBD 1:1 cannabis extract in this application and is to be used in the sublingual preparations.

Preparation of Sublingual Mucoadhesive Tablets Containing THC/CBD 1:1 Cannabis Extract and Buprenorphine

Mucoadhesive Sublingual Tablets Preparation:

Blend the THC/CBD 1:1 cannabis extract, (200 gr of the 70% w/w mixture) with buprenorphine HCl 50 gr, 800 gr mannitol/lactose 1:1 mixture, 30 gr PVP K-30, 20 gr Vitamin E TPGS, granulate with 600 gr ethanol USP and then dry for 45 minutes in a Glatt fluidized bed dryer, at inlet temp 50 deg C.

Mix the dry mixture with an additional amount of 200 gr mannitol, 10 gr sodium citrate & citric acid, 3 gr lemon flavor, 10 gr carbomer 934, 20 gr corn starch and 10 gr magnesium stearate.

Prepare sublingual cannabis extract tablets containing 10 mg of THC/CBD 1:1 and 2 mg buprenorphine HCl.

### Example 5

Preparation of Sublingual Mucoadhesive Film Containing THC/CBD 1:1 Cannabis Extract

Prepare cannabis API using the procedure described in Example No. 1.

Mix the THC/CBD 1:1 cannabis extract (100 gr) with 20 gr Vitamin E TPGS, 10 gr crystalline menthol, 500 gr of wet mass of hydroxypropylmethylcellulose mixed with Polyox WRS N-10, then extrude, dry and cut to a thin film containing 10 mg THC&CBD 1:1.

### Example 6

Preparation of the THC/CBD 1:1 Cannabis Extract

Using cannabis Sativa clone containing about equal amounts of THC (tetrahydrocannabinol) and CBD (cannabidiol), grown under GAP conditions as determined by WHO as source of the botanical extract. Dry, mill and then extract the plant by using supercritical fluid method (CO2) extraction, or non polar extraction with butane. Concentrate the botanical extract to about 70% w/w concentration of CBD &THC (which are in ratio of 1:1). This THC/CBD 1:1 70% extract is defined as the THC/CBD 1:1 cannabis extract in this application and is to be used in the sublingual preparations.

Preparation of Sublingual Mucoadhesive Tablets Containing THC/CBD 1:1 Cannabis Extract

Mucoadhesive Sublingual Tablets Preparation:

Blend the THC/CBD 1:1 cannabis extract (200 gr of the 70% w/w mixture) together with 800 gr mannitol/lactose 1:1 mixture, 30 gr PVP K-30, 20 gr Vitamin E TPGS, granulate with 600 gr ethanol USP and then dry for 45 minutes in a Glatt fluidized bed dryer, at inlet temp 50 deg C.

Mix the dry mixture with an additional amount of 200 gr mannitol, 10 gr sodium citrate & citric acid, 3 gr lemon flavor, 5 gr BHT, 5 gr BHA, 10 gr carbomer 934, 20 gr corn starch and 10 gr magnesium stearate.

Prepare sublingual THC/CBD 1:1 cannabis extract tablets containing 10 mg of THC/CBD 1:1 from the above mixture.

One exemplary embodiment of a tablet formulation of the inventive techniques:

| Ingredient for tablets | Mass |
|---|---|
| Cannabis extract 15-20 mg/tab | 15-20 mg/tab |
| Mannitol | |
| Lactose | |
| Citric acid | |
| Sodium citrate | |
| PVP | |
| Carbomer | |
| Starch | |
| Menthol | |
| Vitamin E TPGS | |
| Aspartame | |
| BHT | |
| Mg Stearate | |

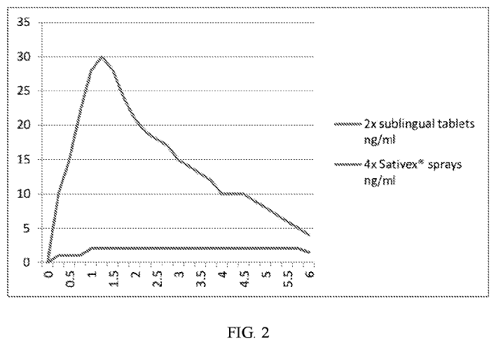

Table 2 below shows the data for the first six hours from a comparison of THC plasma levels following administration of two sublingual tablets prepared according to the exemplary embodiments, 10 mg THC, 10 mg CBD versus 4 sprays of Sativex, 10.8 mg THC, 10 mg CBD. Plasma levels are presented in ng/ml. The sublingual tablets of the exemplary embodiment do not contain alcoholic residues and are non-irritable to oral and buccal mucosa. The data in Table 2 below generates the graph in FIG. 2.

TABLE 2

| | 2x sublingual | 4x Sativex ® |
|---|---|---|
| Time (hrs) | tablets ng/ml | sprays ng/ml |
| 0 | 0 | 0 |
| 0.25 | 10 | 1 |
| 0.5 | 15 | 1 |
| 0.75 | 22 | 1 |
| 1 | 28 | 2 |
| 1.25 | 30 | 2 |
| 1.5 | 28 | 2 |
| 1.75 | 24 | 2 |
| 2 | 21 | 2 |
| 2.25 | 19 | 2 |
| 2.5 | 18 | 2 |
| 2.75 | 17 | 2 |
| 3 | 15 | 2 |
| 3.25 | 14 | 2 |
| 3.5 | 13 | 2 |
| 3.75 | 12 | 2 |
| 4 | 10 | 2 |
| 4.25 | 10 | 2 |
| 4.5 | 10 | 2 |
| 4.75 | 9 | 2 |
| 5 | 8 | 2 |
| 5.25 | 7 | 2 |
| 5.5 | 6 | 2 |
| 5.75 | 5 | 2 |
| 6 | 4 | 1.5 |

TABLE 3
Plant cannabinoids

Cannabigerol-type (CBG): Cannabigerol (E)-CBG-C5; Cannabigerol monomethyl ether (E)-CBGM-C5 A;
Cannabinerolic acid A (Z)-CBGA-C5 A; Cannabigerovarin (E)-CBGV-C3; Cannabigerolic acid A (E)-CBGA-C5 A;
Cannabigerolic acid A monomethyl ether (E)-CBGAM-C5 A; Cannabigerovarinic acid A (E)-CBGVA-C3 A
Cannabichromene-type (CBC): (±)-Cannabichromene CBC-C5; (±)-Cannabichromenic acid A CBCA-C5 A;
(±)-Cannabivarichromene, (±)-Cannabichromevarin CBCV-C3; (±)-Cannabichromevarinic acid A CBCVA-C3 A
Cannabidiol-type (CBD): (−)-Cannabidiol CBD-C5; Cannabidiol momomethyl ether CBDM-C5; Cannabidiol-C4 CBD-C4;
(−)-Cannabidivarin CBDV-C3; Cannabidiorcol CBD-C1; Cannabidiolic acid CBDA-C5; Cannabidivarinic acid CBDVA-C3
Cannabinodiol-type (CBND): Cannabinodiol CBND-C5; Cannabinodivarin CBND-C3
Tetrahydrocannabinol-type (THC): Δ9-Tetrahydrocannabinol [Δ9-THC-C5]; Δ9-Tetrahydrocannabinol-C4 [Δ9-THC-C4];
Δ9-Tetrahydrocannabivarin [Δ9-THCV-C3]; Δ9-Tetrahydrocannabiorcol [Δ9-THCO-C1]; Δ9-Tetrahydro-cannabinolic
acid A [Δ9-THCA-C5 A]; Δ9-Tetrahydro-cannabinolic acid B [Δ9-THCA-C5 B]; Δ9-Tetrahydro-cannabinolic acid-C4 A
and/or B [Δ9-THCA-C4 A and/or B]; Δ9-Tetrahydro-cannabivarinic acid A [Δ9-THCVA-C3 A]; Δ9-Tetrahydro-cannabiorcolic
acid A and/or B [Δ9-THCOA-C1A and/or B (−)-Δ8-trans-(6aR,10aR); Δ8-Tetrahydrocannabinol [Δ8-THC-C5];
(−)-Δ8-trans-(6aR,10aR)-Tetrahydrocannabinolic acid A [Δ8-THCA-C5 A]; (−)-(6aS,10aR)-Δ9-Tetrahydrocannabinol
[(−)-cis-Δ9-THC-C5]
Cannabinol-type (CBN): Cannabinol [CBN-C5]; Cannabinol-C4 [CBN-C4]; Cannabivarin [CBN-C3]; Cannabinol-C2 [CBN-C2];
Cannabiorcol [CBN-C1]; Cannabinolic acid A [CBNA-C5 A]; Cannabinol methyl ether [CBNM-C5]
Cannabitriol-type (CBT): (−)-(9R,10R)-trans-Cannabitriol [(−)-trans-CBT-C5]; (+)-(9S,10S)-Cannabitriol [(+)-trans-CBT-C5];
(±)-(9R,10S/9S,10R)-Cannabitriol [(±)-cis-CBT-C5]; (−)-(9R,10R)-trans-10-O-Ethyl-cannabitriol [(−)-trans-CBT-OEt-C5];
(±)-(9R,10R/9S,10S)-Cannabitriol-C3 [(±)-trans-CBT-C3]; 8,9-Dihydroxy-Δ6a(10a)-tetrahydrocannabinol [8,9-Di-OH-CBT-C5];
Cannabidiolic acid A cannabitriol ester [CBDA-C5 9-OH-CBT-C5 ester]; (−)-(6aR,9S,10S,10aR)-9,10-Dihydroxy-hexahydrocannabinol
Cannabiripsol-C5: (−)-6a,7,10a-Trihydroxy-Δ9-tetrahydrocannabinol; (−)-Cannabitetrol
10-Oxo-Δ6a(10a)-tetrahydrocannabinol [OTHC]
Cannabielsoin-type (CBE): (5aS,6S,9R,9aR)-Cannabielsoin [CBE-C5]; (5aS,6S,9R,9aR)-C3-Cannabielsoin [CBE-C3];
(5aS,6S,9R,9aR)-Cannabielsoic acid A [CBEA-C5 A]; (5aS,6S,9R,9aR)-Cannabielsoic acid B [CBEA-C5 B];
(5aS,6S,9R,9aR)-C3-Cannabielsoic acid B [CBEA-C3 B]
Cannabiglendol-C3: OH-iso-HHCV-C3
Dehydrocannabifuran [DCBF-C5]
Cannabifuran [CBF-C5]
Isocannabinoids: (−)-Δ7-trans-(1R,3R,6R)-Isotetrahydrocannabinol; (±)-Δ7-1,2-cis-(1R,3R,6S/1S,3S,6R)-Isotetrahydro-cannabivarin;
(−)-Δ7-trans-(1R,3R,6R)-Isotetrahydrocannabivarin
Cannabicyclol-type (CBL): (±)-(1aS,3aR,8bR,8cR)-Cannabicyclol [CBL-C5]; (±)-(1aS,3aR,8bR,8cR)-Cannabicyclolic acid A
[CBLA-C5 A]; (±)-(1aS,3aR,8bR,8cR)-Cannabicyclovarin [CBLV-C3]
Cannabicitran-type (CBT): Cannabicitran [CBT-C5]
Cannabichromanone-type (CBCN): Cannabichromanone [CBCN-C5]; Cannabichromanone-C3 [CBCN-C3];
Cannabicoumaronone [CBCON-C5]

1.-27. (canceled)

28. A composition for the sublingual delivery of cannabinoid active ingredients comprising a therapeutically effective dose of at least one cannabinoid, an emulsifying and penetration enhancing solubilizer, a permeation enhancer, and a mucoadhesive polymer, wherein said composition is administered sublingually in the form of tablet or film;
further wherein said permeation enhancer comprises menthol in an amount of 1-50 mg per dose.

29. The sublingual delivery composition of claim 28, wherein said cannabinoid active ingredient is characterized by at least one of the following
a. comprising THC and CBD in a ratio of 5:95;
b. comprising THC in an amount between 0.5 and 50 mg;
c. comprising CBD in an amount between 0.5 and 50 mg.

30. The sublingual delivery composition of claim 28, wherein said emulsifying permeation enhancing solubilizer comprises at least one of Vitamin E TPGS and similar amphiphilic materials with enhancing characteristics like fatty acids derivatives, terpenes, cationic surfactants, anionic surfactants, short chain glycerides and derivatives thereof, cyclodextrine derivatives, and polyols.

31. The sublingual delivery composition of claim 28, wherein said emulsifying enhancing solubilizer comprises Vitamin E TPGS.

32. The sublingual delivery composition of claim 28, wherein said permeation enhancer is selected from the group consisting of menthol, piperine, camphor, Vitamin E TPGS.

33. The sublingual delivery composition of claim 28, wherein said mucoadhesive polymer comprises carbomer, polyethylene oxide, polyvinylpyrrolidone, cellulose based polymers, HPMC, HPC or chitosan.

34. The sublingual delivery composition of claim 28, further comprising at least a second active ingredient.

35. The sublingual delivery composition of claim 34, wherein said second active ingredient comprises one or more bupropion, buprenorphine, naloxone and methadone, one or more cannabinoids and derivatives thereof.

36. The sublingual delivery composition of claim 28, wherein said cannabinoid active ingredient has been extracted by an extraction method comprising either supercritical fluid extraction with Carbon Dioxide (CO₂) or extraction with a non-polar solvent.

37. The sublingual delivery composition of claim 28, wherein said cannabinoid active ingredient is selected from a group consisting of THCA and CBDA.

38. The sublingual delivery composition of claim 28, wherein said composition is preferentially adherent to oral mucosa or buccal mucosa.

39. The sublingual delivery composition of claim 28, wherein said polymer exhibits muco-adhesion and comprises polyethylene oxide, carbomer, polyvinylpyrrolidone, cellulose-based polymers or chitosan, in an amount ranging from 2-100 mg per dosage form.

40. The composition of claim 28, further comprising a solubilizer which is also a cannabinoid active ingredient stabilizer, enhancer and anti-oxidant.

41. The composition of claim 28, further comprising an antioxidant comprising BHT, BHA or mixtures thereof.

42. The sublingual delivery composition of claim 28, wherein said menthol is dissolved in oil or is in crystal form.

43. A method of treatment of opioid addiction and dependency by administration to a patient in need thereof of a therapeutically effective amount of a composition of claim 28 and optionally an additional active ingredient.

44. The sublingual delivery composition of claim 43, wherein said additional active ingredient comprises at least one of a benzodiazepine, buprenorphine, naloxone, methadone, bupropion and other opioid withdrawal compounds and/or other addiction substances.

45. The sublingual delivery composition of claim 43, wherein said optionally additional active ingredient works synergistically with THC and CBD or other cannabis components or their composition.

46. A sublingual composition comprising a cannabinoid active ingredient in a mucoadhesive dosage form, comprising sublingual tablets or films and having greater bioavailability than an alcohol-based oral spray having 5.2 mg/per spray of cannabinoid active ingredients.

47. The sublingual composition of claim 46 having a plasma profile with an AUC at least 150% greater than an alcohol-based oral spray.
